(19) United States
(12) Patent Application Publication
(10) Pub. No.: US 2015/0352265 A1
(43) Pub. Date: Dec. 10, 2015

(54) BIOCOMPATIBLE COATING COMPOSITION

(71) Applicants: Narayana GARIMELLA, Herndon, VA (US); Zhongjun WU, Marriottsville, MD (US); Bartley GRIFFITH, Gibson Island, MD (US)

(72) Inventors: Narayana GARIMELLA, Herndon, VA (US); Zhongjun WU, Marriottsville, MD (US); Bartley GRIFFITH, Gibson Island, MD (US)

(21) Appl. No.: 14/759,252

(22) PCT Filed: Jan. 6, 2014
(86) PCT No.: PCT/US14/10391

§ 371 Date: Jul. 6, 2015

Related U.S. Application Data
(60) Provisional application No. 61/749,495, filed on Jan. 7, 2013.

(51) Int. Cl. A61L 33/00; A61L 33/04; A61L 33/06

(52) U.S. Cl. CPC A61L 33/0023; A61L 33/064; A61L 33/04; A61L 33/0029; A61L 33/0076; A61L 2420/02; A61L 2420/08; A61L 2420/06; A61L 2300/608; A61L 2300/42; A61L 2300/236; A61L 2300/208

(57) ABSTRACT

Provided herein are hemo-compatible, anti- and non-thrombogenic, heparin-based bioactive coatings for a surface to be contacted with blood, such as a surface of an oxygenator device (e.g., a Hollow Fiber Membrane (HFM) surface) or an artificial lung, which coatings include a quaternary ammonium salt and heparin complex (QUAT). According to example embodiments, the surface is treated with polyvinylpyrrolidone (PVP), followed by a coating layer of said quaternary ammonium salts and heparin complex (QUAT) to form a PVP-QUAT coating. According to other example embodiments, anionic functional groups are created on the one or more surfaces of the HFM by modifying the surface of the HFM using ionic complexes dissolved in a solvent mixture that includes major quantity of alcohol along with a minor quantity of organic dissolving agents. Also provided are methods of making the provided coatings, methods of coating a surface, oxygenator devices and artificial lungs that have been coated, and kits that include such coatings.

### RELATED APPLICATION

This application is a PCT application claiming the benefit of priority to U.S. Provisional Application No. 61/749,495 filed on Jan. 7, 2013, the entire contents of which are hereby incorporated by reference.

### STATEMENT OF FEDERALLY SPONSORED RESEARCH

This invention was made with government support under Grant Numbers R01 IIL082631 and R42 IIL084807 awarded by the National Institutes of Health. The government has certain rights in the invention.

### FIELD

The present invention generally relates to biocompatible coating compositions. More specifically, the present invention relates to hemo-compatible, anti- and non-thrombogenic, heparin based bioactive coatings for permeable membrane surfaces.

### BACKGROUND

Next to cardiovascular illness and cancer, lung disease threatens to be the third killer in the United States. (Lung disease data 2008. American Lung Association. http://www.lungusa.org/about-us/publications/ (accessed July 2011).) Hundreds of thousands of deaths in the United States each year are attributed to pulmonary causes, costing hundreds of billion dollars. Id. At present, irreversible and chronic lung disease can only be treated by lung transplantation (www.ISHLT.org). Adult respiratory distress syndrome (ARDS) afflicts 150,000 U.S. patients yearly with morbidity of 30-50% (Demling, R., The modern version of adult respiratory distress syndrome. Ann Rev Med, 1995. 46: p. 193-202; Zwischenberger J B, C. S., Alpard S K, et al., Percutaneous extracorporeal arteriovenous CO₂removal for severe respiratory failure. Ann Thorac Surg, 1999. 68: p. 181-7). The artificial lung might be reasonably introduced clinically as a temporary bridge to transplantation and as acute short-term therapy for ARDS.

Polymeric Hollow Fiber Membranes (HFMs) are an integral part of membrane oxygenators and have greatly revolutionized blood oxygenation systems. HFMs however, are prone to protein adsorption, platelet adhesion and formation and adhesion of thrombotic depositions. Therefore, blood flow in contact with synthetic HFMs has the potential to activate cellular and molecular components of coagulation and inflammation. Artificial blood contacting surfaces cause material-induced blood trauma through the phenomena of contact activation, protein adsorption and elemental activation and adhesion that can ultimately lead to inflammation and adverse clinical responses (FIG. 1). In particular, blood oxygenators and artificial lungs use larger surface areas and extended dwelling times to achieve high gas exchange rates compared to any other blood contacting artificial organs. This is problematic when the patient requires long term support. One solution to minimize thrombosis is to maintain systemic anticoagulation by introducing anticoagulants such as heparin into the patient's blood. However, excessive anticoagulation can lead to the risk of bleeding.

A second solution is to provide blood-contacting surfaces of oxygenators and artificial lungs with biocompatible coatings to reduce the required amount of systemic anticoagulation and to minimize surface induced thrombosis (or) blood activation and clotting due to surfaces. Many studies have confirmed that both heparin based bioactive surface modifications and non-heparin based biopassive coating methods provide beneficial effects. However, the anticoagulation properties of many of these coatings only lasts a short period of time and cannot be used for long term support.

Accordingly, there is a need for non-thrombogenic biocompatible coatings that maintain their structural integrity and non-thrombogenic properties during extended use.

### SUMMARY p According to non-limiting example embodiments, the invention provides non-thrombogenic bio-compatible coatings.

In example embodiments, Quaternary Ammonium Salts and Heparin Complex (QUAT) coatings are provided. Exemplary embodiments of QUAT coatings provided herein include surface-modification of HFMs or other membranes or surfaces using ionic complexes dissolved in a solvent mixture that includes a major quantity of alcohol along with a minor quantity or quantities of organic dissolving agents such as Tetrahydrofuran (THF), Toluene, Petroleum ether, etc. In other embodiments, polyvinylpyrrolidone (PVP) is primarily coated followed by a later coating of quaternary ammonium salts and heparin complex (PVP-QUAT). PVP-QUAT coatings are intended to induce hybridizing non-thrombogenic effects of PVP and anti-thrombogenic effects of heparin during blood contact with the surfaces.

Further example embodiments are directed to methods for preparing and applying the coatings to a surface, such as a permeable membrane surface. Further provided are oxygenator devices and artificial lungs that have been coated, and kits that include such coatings.

### BRIEF DESCRIPTION OF DRAWINGS

Non-limiting example embodiments are described herein, with reference to the following accompanying Figures:

FIG. 1 is a flowchart that depicts consequences of blood interaction with hollow fiber membranes of oxygenators.

FIG. 2 illustrates the principle of oxygenation through hollow fiber membranes. FIG. 2A shows oxygen and carbon dioxide flow through a hollow fiber membrane and through series of such membranes as a part of mat. FIG. 2B shows surface and cross-sectional features of hollow fiber membranes under photographic, optical and electron microscopic levels of magnification.

FIG. 3 shows schematics of immobilized surfaces to maintain blood compatibility of a heparin coating. FIG. 3A is a schematic that indicates the principle through which heparinized surfaces maintain blood compatibility. FIG. 3B depicts the principle of coating Quaternary ammonium salt and heparin over the surface of a hollow fiber membrane. FIG. 3C depicts the principle of coating PVP, Quaternary ammonium salt and heparin over the surface of a hollow fiber membrane.

FIG. 4 is a graph that depicts levels of immobilized heparin on the Corline heparin surface (CHS) coated fibers, Quat(s)-heparin complex coated fibers, and the PVP and Quat(s)-heparin complex coated fibers.

FIG. 5 shows amounts of fibrinogen adsorbed on the surfaces of HFMs.

FIG. 6 shows quantities of platelets adhered on the surfaces of HFMs.

FIG. 7 depicts the scanning electron microscopy observation of in-vitro blood-contacted surfaces of current coatings along with the non-coated and some commercially coated HFM surfaces.

FIG. 8 shows the amounts of thrombus estimated from the surfaces of HFMs of FIG. 7.

FIG. 9 shows example structures of quaternary ammonium halides in accordance with example embodiments.

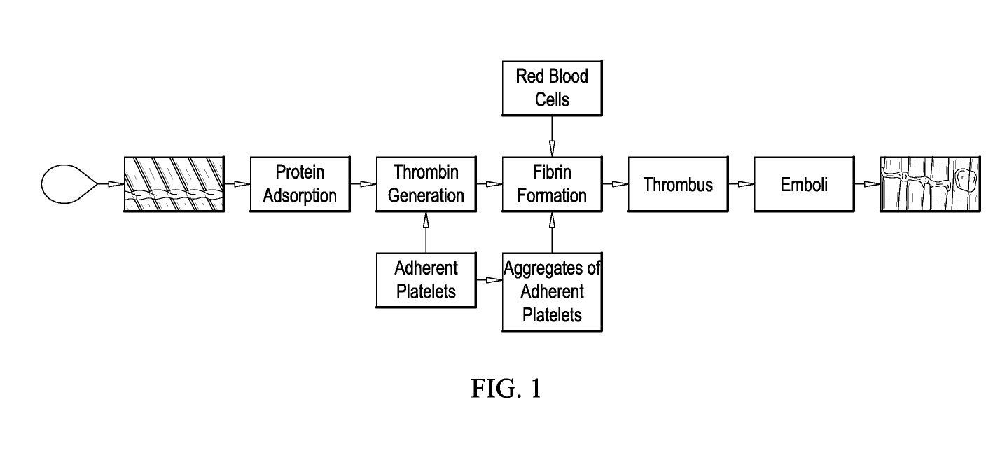

### DETAILED DESCRIPTION

The present invention is drawn to non- and anti-thrombogenic, bio-compatible coatings and methods for making and using such coatings. Also provided are oxygenator devices and artificial lungs that have been coated, and kits that include such coatings.

Additional aspects, advantages and/or other features of example embodiments of the invention will become apparent in view of the following detailed description, taken in conjunction with the accompanying drawings. It should be apparent to those skilled in the art that the described embodiments provided herein are merely exemplary and illustrative and not limiting. Numerous embodiments of modifications thereof are contemplated as falling within the scope of this disclosure and equivalents thereto.

While the example embodiments are described to be used in conjunction with hollow fiber membranes in blood oxygenation systems, it should be understood that these coatings may be used for other purposes and therefore the present invention is not limited to such applications. In view of the teachings provided herein, one having ordinary skill in the art would recognize other applications for which the coatings of the present invention could be used. Thus, one having ordinary skill in the art would be able to use the coatings and methods of the present invention in other applications. Accordingly, these alternative uses are intended to be part of the present invention.

All publications mentioned in this specification are indicative of the level of those skilled in the art to which the invention pertains. All patents and publications herein are incorporated by reference to the same extent as if each individual publication was specifically and individually indicated as having been incorporated by reference in its entirety.

In describing example embodiments, specific terminology is employed for the sake of clarity. However, the embodiments are not intended to be limited to this specific terminology. Unless otherwise noted, technical terms are used according to conventional usage.

As used herein, “a” or “an” may mean one or more. As used herein “another” may mean at least a second or more. Furthermore, unless otherwise required by context, singular terms include pluralities and plural terms include the singular.

As used herein the terms “coating”, “coating composition” and “composition” are used someone interchangeably with regard to a coating composition to be applied over at least one surface of e.g. a membrane, such as hollow fiber membrane that forms part of an oxygenator. Such application of the coating may be over a primer or other layer or treatment that is first applied. Thus the coating may not directly touch the surface. Also, the coating may or may not completely coat a particular surface or portion of the surface. Coatings provided herein may be very thin (e.g., less than 100 nanometers thick) and transparent. With direct visual observation, there may not appear to be any difference between coated and uncoated hollow fiber membrane surfaces. Therefore, coating is regarded as a gas-permeable and blood-compatible interface between blood and membrane surface.

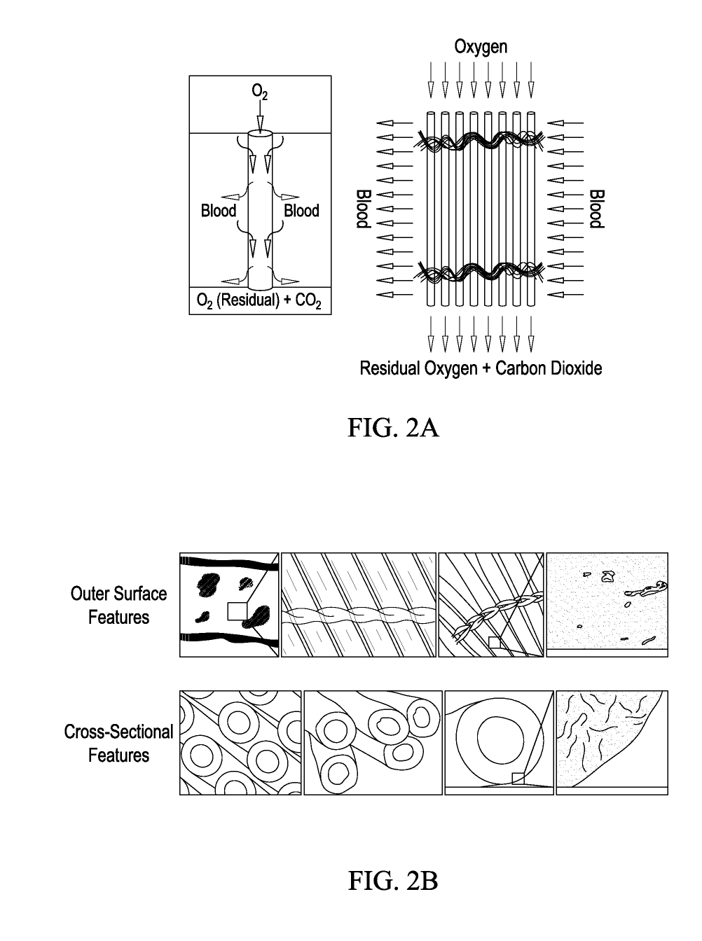

As used herein, the terms “Hollow Fiber Membranes” or “HFMs” are used interchangeably to refer to small, semi-permeable capillary tubes that enable blood oxygenation by the processes of diffusion of oxygen into blood and of carbon dioxide into gas phases in contact with them. An HFM surrounded by flowing blood with oxygen flowing through its inner lumen describes the basic principle of oxygenation as shown in FIG. 2A. Due to the counter current action of blood and oxygen with the aid of diffusion, carbon dioxide along with the residual oxygen leaves out the bottom. HFMs may be bundled together to maintain integrity and to increase the efficiency of blood oxygenation.

A series of optical and scanning electron microscopy images illustrating outer surface features and cross-sectional features of example HFMs are shown in FIG. 2B. HFMs are prone to protein adsorption, platelet adhesion and formation and adhesion of thrombotic depositions. Thrombotic depositions inhibit gas transfer and therefore limit the duration of their use. The present invention provides biocompatible coatings to reduce the degree of thrombotic depositions, which allows for extended use of oxygenation systems.

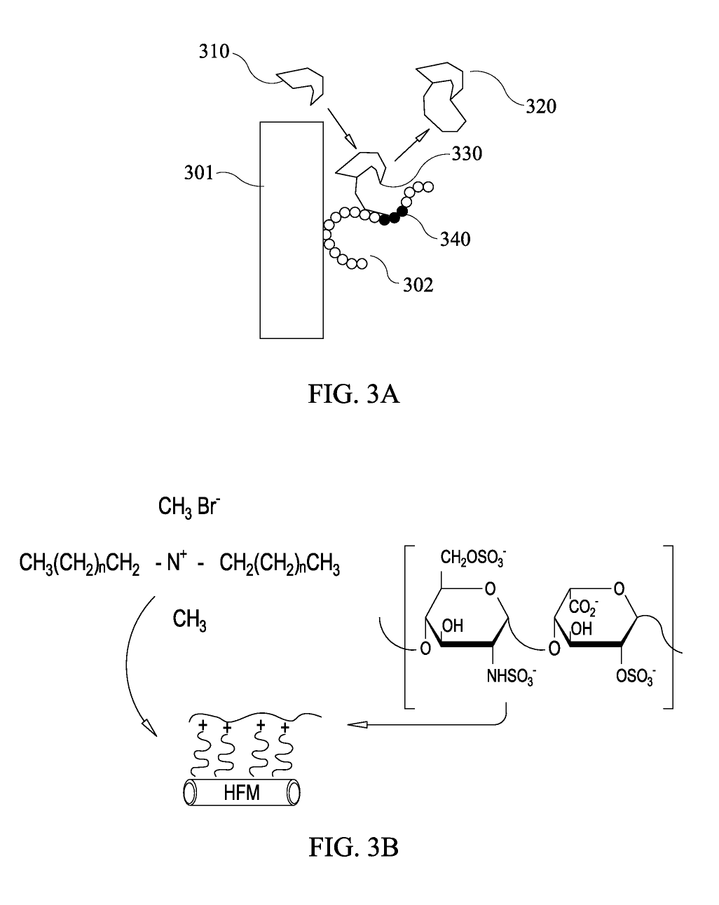

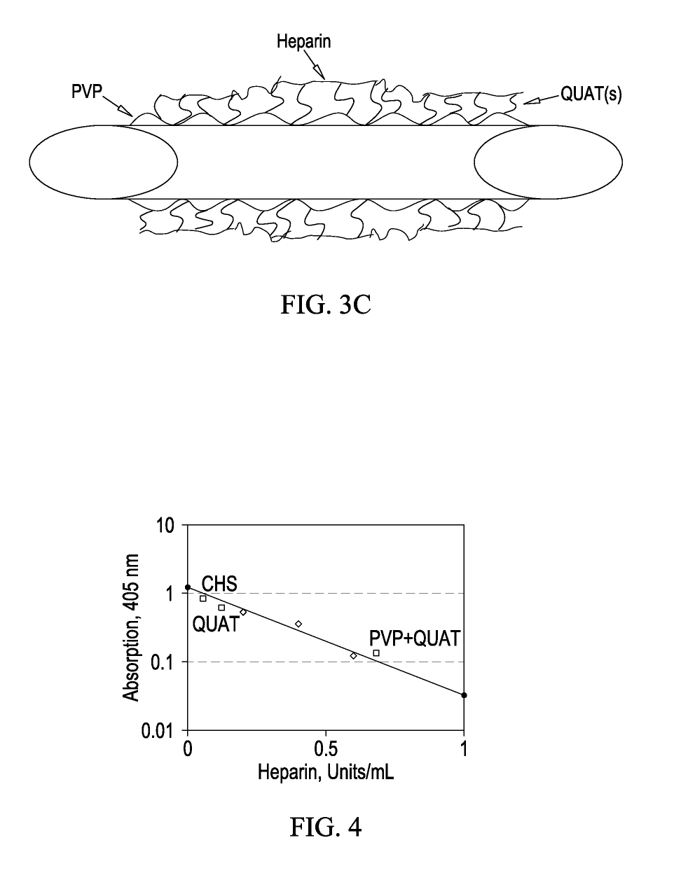

FIG. 3A indicates the principle through which heparinized surfaces maintain their blood compatibility on a substrate, 301. Heparin, 302, which is immobilized on substrate, 301, provides active AT III sites, 340. The potential thrombogenic agent of Thrombin, 310, is mitigated by antithrombin, 330, as they both bind together to make a neutralized resulting of thrombin-antithrombin (AT III) complexes (TAT), 320. Thereby the thrombin, 310, is inactivated when the TAT complexes bind to the active AT III sites, 340.

### Coating Compositions

Non-limiting example embodiments of the present invention provide for a Quaternary Ammonium Salts and Heparin Complex (QUAT) coating composition. In particular, provided herein are biocompatible coating compositions that include hemo-compatible, heparin-based bioactive coatings comprising a quaternary ammonium salt and heparin complex (QUAT). As depicted e.g., in FIG. 9, the structure may include specific linear quaternary ammonium halides with their positive nitrogen ions bonded ionically with some negative ions of the heparin. Some of the positive nitrogen ions of quaternary ammonium halides are bonded with the negative ions of the functional groups attained on the surfaces of membranes. The coatings may be anti- and non-thrombogenic.

Other example embodiments provide for PVP primed Quaternary Ammonium Salts and Heparin Complex (PVP-QUAT) coatings. In particular, examples of these coating compositions include a coating layer of polyvinylpyrrolidone (PVP), followed by a coating layer of said quaternary ammonium salts and heparin complex (QUAT) to form a PVP-QUAT coating.

### Creation of Anionic Functional Groups on The Surfaces of HFMs:

Contacting the outer surfaces of PMP-Oxyplus HFMs of 1 cm² in size in plain Methanol solvent (or) in a solvent mixture of 70 vol. % Methanol and 30 vol. % of Tetrahydrofuran (THF) results in a maximum density of negative ions on the surfaces of HFMs. The presence and density of these sites (or negative ions) were confirmed by adsorbing Toluidine Blue (cationic or +ve ionic) dye on the HFMs. Independent solutions of Toluidine Blue were prepared by dissolving it in the range of 0.005 to 0.05 wt. % in 0.01N HCl solution containing 0.2 wt. % NaCl. It was confirmed that, 1 cm² surface area of HFMs were capable of adsorbing the maximum of 0.05 wt. % of Toluidine Blue from 1 ml of solution. Because Toluidine Blue is a cationic dye with analogues characteristics as cationic quaternary ammonium halides with respect to positively charged Nitrogen ions, Toluidine Blue was taken as a standard in estimating the amount of quaternary ammonium halides required for the surface immobilization.

The present inventors have found that alcohol or alcohols alone have somewhat limited dissolving-ability for the complex of Heparin and Quaternary ammonium halides. The present inventors have found that THF or toluene or other organic solvents herein will enhance the dissolving ability. Thus, according to non-limiting example embodiments, a solvent mixture having 70-100 vol. % methanol (or other alcohol or mixture of alcohols including methanol, ethanol and/or propanol) and 0-30 vol. % of THF may be used. According to further non-limiting example embodiments, the solvent mixture may include 70-80 vol. % Methanol (or other alcohol or mixture of alcohols including methanol, ethanol and/or propanol) and 20-30 vol. % of THF. The solvent mixture may optionally also include the presence of additional minor organic agents such as toluene, petroleum ether, ether, benzene etc . . . in an amount of up to 10% by volume. With an increase in the additions such as toluene, ether and other organic reagents, there may be adverse effects on the integrity of the hollow fiber membranes. Therefore, their amount in the final coating solution may be limited to below 10%. Thus, according to example embodiments, the mixture may be predominantly alcohol, with 100% alcohol being the limit of the solvent; additional reagent being mainly intended as a dissolving agent for the complex of heparin and quaternary ammonium salts. THF was found to have fewer adverse effects on the membranes and its concentration may be up to 30% within the alcohol solvent.

### Methods for Synthesizing Coating Compositions

### Synthesis of Quaternary Ammonium Salt(s) and Heparin Complex (Quat(s)-Heparin Complex)

A first solution of 5 grams of heparin dissolved in 100 milliliters of deionized water was mixed in a drop-wise fashion with a second solution containing hydrophobic Quaternary ammonium salt or a mixture of one or more hydrophobic Quaternary ammonium salts with or without the mixture of one or more hydrophilic Quaternary ammonium salts dissolved in alcohol. That is, the second solution may include for example, quaternary halides dissolved in alcohol. These halides of the second solution can be either one or more. But, according to example embodiments, a predominant amount of the solution should comprise hydrophobic characteristics.

The salts can be any from the long-chain linear Quaternary aliphatic alkylammonium type with at least one radical being a long-chain aliphatic group from C₇H₁₅ to C₁₈H₃₇ either plain or substituted (Rahn, Otto, and William P. Van Eseltine. “Quaternary ammonium compounds.” Annual Reviews in Microbiology 1, no. 1 (1947): 173-192). The amounts of salts in the methanol solution were maintained such that for every tetrasaccharide unit of the heparin there exists 5 to 15 amine sites of cationic linear quaternary ammonium halides (Falb, R. D., Grode, G. A., Takahashi, M. T., & Leininger, R. I. (1968). Characteristics of Heparinized Surfaces. Interaction of Liquids at Solid Substrates, Alexander, A L, eds. American Chemical Society, Washington, D.C).

Thus, according to non-limiting example embodiments, provided herein are methods for preparing non-thrombogenic bio-compatible coatings, which include mixing a solution of heparin dissolved in water with a solution that includes hydrophobic Quaternary ammonium salts, or a mixture comprising one or more hydrophobic Quaternary ammonium salts with or without a mixture of one or more hydrophilic Quaternary ammonium salts dissolved in alcohol. According to further embodiments, the salts may include one or more long-chain linear Quaternary aliphatic alkylammonium salts with at least one radical being a long-chain aliphatic group from C₇H₁₅ to C₁₈H₃₇ either plain or substituted. The mixing may be performed in a drop-wise fashion.

Solvents for dissolving heparin and (or) Quat (s) may either be replaced or modified with other types of organic or inorganic solvents such as buffer solutions, dichloromethane, tetrahydrofuran, toluene, petroleum ether, benzene, and etc. . . . Methanol was identified to be a preferred (but not limiting) solvent for dissolving quaternary ammonium halides. In addition, Methanol has no damaging influence on the mechanical integrity and structure of the HFMs. According to example embodiments, other alcohols or mixtures of alcohols including methanol, ethanol and/or propanol may be used. Solvents such as Tetrahydrofuran, Acetone, Toluene, Naphtha, n-heptane, cyclohexane, n-hexane, Ether, Petroleum-Ether, Benzene, etc . . . may be preferred, but are not limiting, as additional possible organic dissolving agents for the complex of quaternary ammonium halides and heparin. However, the lowest possible quantities of these solvents may be used while preparing the final coating solution because excessive organic solvents may damage the HFMs chemically, physically and mechanically. By way of example, according to example embodiments, Tetrahydrofuran may be confined to be for example, 30 vol. % maximum of the total coating solution. If toluene is used, according to example embodiments, it may not exceed 10 vol. % of the coating solution. If petroleum ether is used, according to example embodiments, it may not exceed 5 vol. % of the total coating solution.

According to Example embodiments, provided herein are methods of coating a membrane surface, such as a Hollow Fiber Membrane (HFM) surface, that include creating anionic functional groups on one or more surfaces of said HFM. Also provided herein are anionic functional groups created on the one or more surfaces of an HFM by modifying the surface of said HFM using ionic complexes dissolved in a solvent mixture. The solvent mixture may include major quantity of alcohol along with a minor quantity of organic dissolving agents. According to non-limiting example embodiments, an organic dissolving agent comprises one or more of Tetrahydrofuran (THF), Toluene, and Petroleum ether. As indicated above, the solvent mixture may include 70-100 vol. % Methanol and 0-30 vol. % of THF.

The details of quaternary ammonium halides and heparin are presented in Table 1.

TABLE 1
Details of quaternary ammonium salts and heparin
applied to the outer surfaces of Oxyplus PMP-HFMs.

| | | | Salient |
|---|---|---|---|
| Name of the Chemical or | | | Features (or) |
| Constituent | Company | Chemical Formula | Characteristics |
| Dimethyldioctadecyl- | D2779-10G | C38H80NBr | Hydrophobic |
| ammonium Bromide | (Sigma Aldrich) | Molecular Weight: 630.98 | |
| Didodecyldimethyl- | 359025-10G | C26H56NBr | Hydrophilic |
| ammonium Bromide, | (Sigma Aldrich) | Molecular Weight: 462.65 | |
| 98% | | | |
| Tetrakis(decyl)- | 87580-10G | C40H84NBr | Hydrophobic |
| ammonium Bromide | (Sigma Aldrich) | Molecular Weight: 462.65 | |
| Hexadecyltrimethyl- | H6269-100G | C19H42NBr | Hydrophilic |
| ammonium Bromide | (Sigma Aldrich) | Molecular Weight: 462.65 | |
| Heparin, Sodium Salt, | 375095-500KU | Molecular Weight: 15000 | Hydrophilic |
| Porcine Intestinal | EMD chemicals | (approximately) | |
| Mucosa | | | |

During the mixing process, stirring of the later solution was ensured to expose a fresh and high surface area of contact each time whereby a colloidal solution comprising a Quat (s)-Heparin complex and residual particles of heparin-rich Quat (s) and Quat (s)-rich heparin suspended within the solvent combination of water and alcohol. Suspended precipitate and particles of this solution were carefully separated in the form of a dense pellet using a method of centrifugation and aliquot separation. Dense indicates solid form of the polymeric precipitate that descends down the liquid due to centrifugation and gravity effects. Washing of the dense precipitate was carried out with a fresh solvent mixture of deionized water and alcohol each time. After aliquot was discarded, residual liquid from the complex was expelled out using the method of freeze-drying at a vacuum of 10⁻³ mm of Hg. Moisture-free-complex so obtained was stored at below −20° C. This complex was weighed, ground to fine particles and finally dissolved in the solvent combination including 60 to 90 wt. % of alcohol with rest being tetrahydrofuran (THF), toluene, petroleum ether, and/or other reagents.

Thus, according to example embodiments, in the present methods the mixing produces a colloidal solution, which includes a Quat (s)-Heparin complex and residual particles of heparin-rich Quat (s) and Quat (s)-rich heparin suspended within the solvent combination of water and alcohol. The method may further include separating suspended precipitate and particles of this solution in the form of a dense pellet using centrifugation and aliquot separation. Such methods may further include washing the dense precipitate with a fresh solvent mixture of deionized water and alcohol one or more times; and discarding aliquot and expelling residual liquid from the complex.

As indicated above, according to non-limiting example embodiments, in the present methods a complex obtained by the method is stored at below −20° C.

The methods may further include weighing the resulting complex and grinding the complex to fine particles and dissolving the complex in a solvent combination comprising 60 to 90 wt. % of alcohol with rest being at least one reagent. The reagent may include at least one reagent selected from the group consisting of tetrahydrofuran (THF), toluene, and petroleum ether.

### Preparation of Priming Solution:

According to non-limiting example embodiments, a surface to be coated may be first primed with a PVP solution. In particular, a solution including 0.5 to 0.9 wt. % of Poly-N-vinyl-2-pyrrolidone (PVP) dissolved in the alcohol was prepared separately for the application of the initial surface modification and this solution was referred to as ‘priming solution’.

### Methods of Coating a Surface or Device:

Prior to coating a surface or device, test fiber samples of Hollow Fiber Membranes may be coated either directly using the coating solution of Quat (s)-Heparin complex dissolved in alcohol rich solvent or the fibers were first prime-coated with PVP dissolved in alcohol, followed by second coat using the above mentioned coating solution.

Thus, provided herein are methods that include coating a surface with a priming solution comprising PVP and thereafter coating said surface with a QUAT solution comprising quaternary ammonium salts and heparin.

Both priming and complex coating solutions and the activity of the heparin was assessed through in-vitro anti-Factor Xa assay. After ensuring the activity of immobilized heparin, coating an oxygenator device was carried out as per the below described procedure.

The path through which liquid or blood flows and contacts the surfaces of an oxygenator device is called flow-path. It includes inlet, outlet, plastic polycarbonate parts, hollow fiber membranes, magnetically driven metallic impeller and other plastic tubings or parts. It is mainly to describe about outer-lumen of the all hollow fiber membranes those have to remain in contact with blood during clinical or experimental application.

According to non-limiting example embodiments, to coat the blood contacting surface of an oxygenator device (e.g. HFM surfaces), the anionic functional groups were initially achieved on a desired surface by treating the device with plain alcohol for at least 2 minutes. This priming solution was first filled into the device through the blood inlet port of the device. The blood contacting surfaces of the HFMs remained in contact with the solution for a minimum of 1 second and a maximum of 10 minutes, or even more. After that, the liquid was discarded and purged out from the oxygenator device using an inert gas such as Nitrogen.

Rinsing the flow path of the oxygenator device with plain alcohol ensures the removal of free or unadsorbed PVP from it.

The Quat (s)-Heparin complex dissolved in alcohol and THF solution is then filled into the device through the blood inlet port of the oxygenator devices. All the HFMs surfaces to be contacted with blood are completely submerged and wetted in the coating solution for a minimum of 1 second to the maximum of 10 minutes. After that, the coating solution was discarded and purged out from the oxygenator device using inert gas. Rinsing the flow path of the oxygenator device with saline solution ensures not only the removal of free or non-immobilized complex but also gets rid of traces of left-over coating.

Accordingly, further to the above, provided herein are methods that include at least the following: filling a solution comprising 0.5 to 0.9 wt. % of Poly-N-vinyl-2-pyrrolidone (PVP) dissolved in alcohol, through a blood inlet port of an oxygenator device; rinsing with alcohol to remove PVP; filling through the blood inlet port of the oxygenator device, a biocompatible coating composition comprising hemo-compatible, anti- and non-thrombogenic, heparin-based bioactive coatings that includes a quaternary ammonium salt and heparin complex (QUAT) dissolved in alcohol and THF solution, and contacting surfaces to be contacted with blood (such as one or more surfaces of Hollow Fiber Membrane (HFM) in the coating solution for 1 second to 10 minutes. The methods may then include discarding and purging the coating composition out from the oxygenator device e.g., using inert gas; and rinsing the oxygenator device with saline solution.

### Oxygenators and Artificial Lungs

Further embodiments of the present invention provide devices that include surfaces coated with the present coating compositions. In particular, by way of example, provided herein are oxygenator devices that include one or more blood contacting surfaces coated with one or more of the present coatings. Further provided herein are artificial lungs that include one or more blood contacting surfaces coated with one or more of the present coatings.

Also provided herein are kits that include, for example, one or more hemo-compatible, anti- and/or non-thrombogenic, heparin-based bioactive coatings compositions that include a quaternary ammonium salt and heparin complex (QUAT); and at least one additional component selected from the group consisting of instructions for making or using such coatings for example on an oxygenator or artificial lung to be used with a mammal; an additional component of a coating, a device or component that may be used in coating a surface, and/or an oxygenator, an artificial lung, an HFM or other surface to be coated. Kits provided herein may additionally include one or more additional components that may be used in forming or using the present coating compositions.

The present invention also includes the use of one or more hemo-compatible, anti- and/or non-thrombogenic, heparin-based bioactive coatings that include a quaternary ammonium salt and heparin complex (QUAT) as a coating, for example on permeable membranes.

Further provided are methods of treating lung disease in a patient. The methods may include for example coating at least one blood-contacting surface of an oxygenator and/or artificial lungs to be used with a patient having a lung disease, with one or more hemo-compatible, anti- and non-thrombogenic, heparin-based bioactive coatings comprising a quaternary ammonium salt and heparin complex (QUAT). Methods may also include administering to a patient having a lung disease at least one oxygenator and/or artificial lung having one or more hemo-compatible, anti- and non-thrombogenic, heparin-based bioactive coatings that include a quaternary ammonium salt and heparin complex (QUAT), on at least one blood-contacting surface of the at least one oxygenator and/or artificial lung.

Further example methods may include coating at least one blood-contacting surface of an oxygenator and/or artificial lungs to be used with a patient having a lung disease, with one or more hemo-compatible, anti- and non-thrombogenic, heparin-based bioactive coatings comprising a quaternary ammonium salt and heparin complex (QUAT) to reduce the required amount of systemic anticoagulation and to minimize surface induced thrombosis (or) blood activation and clotting due to surfaces.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### EXAMPLES

### Example 1

Quantities of immobilized heparin on a PVP-heparin (PVP-HC) coated surface is compared to a heparin coated surface (HC) and a Corline heparin surface (CHS). 200 μL of AT-III and 25 μL of heparin of 0.5 to 15 μg/mL concentration were mixed and incubated at 37° C. for two minutes. 200 μL of FXₐ were then mixed into the solution and incubated for one minute at 37° C. Next, 200 μL of spectrozyme FXₐ was mixed into the solution and was incubated at 37° C. for five minutes. 200 μL of glacial acetic acid were added and mixed thoroughly. Heparin absorption was then measured at 405 nm which quantified the activity of heparin immobilized over the surfaces of the fibers. FIG. 4 is a standard linear line which plots activities of the known amount of heparin quantities with respect to the absorption readings determined through the spectro-photo-meter technique. Quantities of immobilized heparin from the coated fiber samples of CHS, HC, and PVP-HC are indicated on the standard curve. FIG. 4 demonstrates that PVP-HC has a higher amount of immobilized heparin than HC or CHS samples. This shows PVP has an increasing influence in retaining and maintaining higher activity than heparin alone.

### Example 2

The HFMs that were each coated with a different example coating of the present invention were tested to determine their biocompatibility and ability to retard protein adsorption, platelet adhesion and formation and adhesion of thrombotic depositions. These were then compared to non-coated HFM bundles and other commercially available biocompatible coatings on HFMs. Details of the different coatings are provided in Table 2.

TABLE 2

| Coating | | |
|---|---|---|
| (or) | Type of Coating | |
| Surface-Modification | (Bioactive/Biopassive) | Oxygenator |
| CBAS (Carmeda Bioactive | Bioactive- | CB511 Affinity |
| Surface) | Heparin Based | (Medtronic) |
| X- | Biopassive | Capiox 3CXSX18X |
| | Ampiphilic Polymer Based | (Terumo) |
| Phisio | Biomimetic | D905 DIDECO |
| | Phosphorylcoline Based | EOS M PHISIO |
| | | (Sorin Group Italia, Italy) |
| BioLine ® | Bioactive | BEQ-HMOD 2030 |
| | Heparin Based | (Jostra Quadrox, Maquet |
| | | Cardiopulmonary, Hirrlingen, |
| | | Germany) |
| SafeLine ® | Biopassive | HMOD 2030 |
| | Protein Based | (Jostra Quadrox, Maquet |
| | | Cardiopulmonary, Hirrlingen, |
| | | Germany) |
| CHS (Corline Heparin | Bioactive Heparin Based | — |
| Surface) | | |
| QUAT (or) H-Q (Quaternary | Bioactive Heparin Based | — |
| Ammonium Salt and Heparin | | |
| Complex) | | |
| (In-House Coating) | | |
| PVP-QUAT (or) P-H-Q (In- | Bioactive Heparin Based | — |
| House Coating) | | |
| NON (Non-Coated) | No Coating | — |

### Materials and Methods

HFMs with Coatings of the Present Invention

HFMs with the QUAT coating and PVP-QUAT coating of the present invention were used to compare their efficacy with non-coated HFMs and HFMs coated with other commercially available coatings. The QUAT and PVP-QUAT coated HFMs were prepared using the methods described above. Prior to both PVP-QUAT and QUAT coating processes, the HFM fibers were mechanically occluded at both ends to prevent blood from entering through the inner lumens of the HFMs.

### Commercially Coated and Non-Coated Hollow Fiber Membrane Samples

a. Non-Coated Control (NON) HFM Samples

These HFM samples were un-coated PMP-Oxyplus-Membrana (PMP) (Membrana, Germany). The fibers were mechanically occluded at both ends to prevent blood from entering through the inner lumens of the HFMs.

b. CBAS

The CBAS-coated (Larm O , Larsson R, Olsson P. A new non-thrombogenic surface prepared by selective covalent binding of heparin via a modified reducing terminal residue. Biomat Med Rev Artif Organs 1983;11:161-73). HFMs (Primox, Sorin/Dideco, Mirandola, Modena, Italy) were sectioned from the commercial Medtronic oxygenator. CBAS is a bioactive method of coating in which heparin molecules are covalently attached to surfaces using the process of end-point attachment. This coating is non-leachable and its chemical structure allows the immobilized heparin to preserve its antithrombogenic properties on coated surfaces for extended periods of time (Rahn, Otto, and William P. Van Eseltine. “Quaternary ammonium compounds.” Annual Reviews in Microbiology 1, no. 1 (1947): 173-192). Sectioned sample of fibers were mechanically occluded at both ends to prevent liquid (blood) from entering through the inner lumens of the HFMs.

c. Phisio

The Phisio (Phosphorylcholine)-coated HFMs (Primox, Sorin/Dideco, Mirandola, Modena, Italy, Gunaydin S. Emerging technologies in biocompatible surface modifying additives: quest for physiologic cardiopulmonary bypass. Curr Med Chem 2004; 2: 295-302). were sectioned from the commercial Sorin/Dideco oxygenator. The phosphorylcholine polymer (Biocompatibles International plc, Farnham, Surrey, UK) is designed to mimic the endothelial wall and is chemically inert. No heparin is present. Sectioned sample of fibers were mechanically occluded at both ends to prevent liquid (blood) from entering through the inner lumens of the HFMs.

d. Bioline

The Bioline-coated HFMs (Quadrox, Maquet-Dynamed, Hirrlingen, Germany, H. P. Wendel et al: Oxygenator Thrombosis: Worst Case After Development of an Abnormal Pressure Gradient—Incidence and Pathway; Perfusion 2001, 16, 271-278) were sectioned from Maquet-Oxygenator. The Bioline coating combines polypeptides and heparin. Polypeptides are adsorbed onto the components of the CPB surface. The heparin molecules are attached to the polypeptides via stable covalent bonds and ionic interaction. Sectioned sample of fibers were mechanically occluded at both ends to prevent liquid (blood) from entering through the inner lumens of the HFMs.

e. Safeline

The Safeline-coated HFMs (Quadrox, Maquet-Dynamed, Hirrlingen, Germany, H. P. Wendel et al: Oxygenator Thrombosis: Worst Case After Development of an Abnormal Pressure Gradient—Incidence and Pathway; Perfusion 2001, 16, 271-278) were sectioned from Maquet-Oxygenator. The Safeline is a novel biopassive coating that involves physical refinement with an albumin additive which renders hydrophilic characteristics to the surfaces exposed to blood. Binding of the albumin additive to the surface is achieved through electrostatic and van der Waals forces. This binding is highly stable, and no additive is released to the patient blood during extracorporeal circulation. Sectioned sample of fibers were mechanically occluded at both ends to prevent liquid (blood) from entering through the inner lumens of the HFMs.

f. X-Coating

The X-coated (Lyne Schiel, Steve Burns, Atsuhiko Nogawa, Robert Rice, Takao Anzai, Masaru Tanaka, X-Coating: A New Biopassive Polymer Coating, 2011, 11(2), pp. 8-17) or Poly (2-methoxyethylacrylate) (PMEA)-coated HFMs (Capiox RX25, Terumo, N.J., USA) were sectioned from Capiox SX18X oxygenator. The X-coating has a hydrophobic polyethylene backbone that is adherent to the PVC tubing and has a hydrophilic layer which is in contact with the blood. The hydrophilic water layer prevents surface activation and is designed to be biologically inert. (Ueyama K, Nishimura K, Nishina T, Nakamura T, Ikeda T, Komeda M. PMEA coating of pump circuit and oxygenator may attenuate the early systemic inflammatory response in cardiopulmonary bypass surgery. ASAIO J. 2004 Jul-Aug; 50(4): 369-72.; Ikuta T, Fujii H, Shibata T, Hattori K, Hirai H, Kumano H, Suehiro S. A new poly-2-methoxyethylacrylate-coated cardiopulmonary bypass circuit possesses superior platelet preservation and inflammatory suppression efficacy. Ann Thorac Surg. 2004 May; 77(5): 1678-83). Sectioned sample of fibers were mechanically occluded at both ends to prevent liquid (blood) from entering through the inner lumens of the HFMs.

### In-Vitro Protein (Fibrinogen) Adsorption Assessment of HFMs:

The quantities of fibrinogen adsorbed by the outer surfaces of the HFMs samples were quantified by using Quick Start Bradford protein assay (Bio-Rad). The principle of this assay relies on measuring depletions of fibrinogen from aqueous solution by binding with the dye of coomassie Brilliant Blue G-250.

HFM samples were initially rinsed in 10 mM phosphate-buffered saline (PBS) of pH: 7.4 solution for 30 minutes followed by submerging them within 1 mg/ml of fibrinogen dissolved in one ml of PBS sample solutions at 37° C. for 60 minutes. Quantities of fibrinogen adsorbed by HFMs of each sample was quantified by mixing and reacting 10 μl of sample with 200 μl of dye dissolved in PBS solution and further by measuring the absorption at the wavelength of 595 nm. Comparing the absorption of samples with the absorption of standards through linear equation enables the quantification of fibrinogen content per each HFM sample surface.

Blood Collection and Assessment of in-vitro Hemocompatibility of HFMs:

Fresh whole human blood was collected from healthy consenting volunteers. Blood was anticoagulated with concentrations of heparin being 5 IU in 1 mL of whole blood. The non-coated HFM samples and the samples of surface-modified HFMs with biocompatibility-coatings were labeled as independent samples and were placed in blood-collection tubes of each 3 ml volume capacity. The fibers of each sample were incubated in 3 mL of heparinized blood and rocked for 3 hours at 37° C. on a hematology mixer followed by washing six times with 10 mM phosphate-buffered saline (PBS).

### Total Blood Count Analyses to Determine:

Blood samples were taken from the tube with no-fibers and from the nine tubes in contact with fiber samples. The counts of the blood cells including Platelets, WBCs and RBCs were measured in an automated analyzer (Hemavet).

### Determination of Number of Platelets Adhered on the Surfaces of HFM Samples:

The number of platelets adhered on the fiber samples were determined by a lactate dehydrogenase (LDH) assay method (QuantiChrom LDH Kit, BioAssay Systems, CA, DLDH-100, QuantiChrom™, Lactate Dehydrogenase Kit, Colorimetric Kinetic Determination of Lactate Dehydrogenase Activity, QuantiChrom LDH Kit, BioAssay Systems, CA). As platelets contain LDH, they release LDH into the surrounding liquid medium, where it is identified in higher than normal levels. Therefore, LDH may be taken as a measure to evaluate the adhered platelets to HFMs. This is a non-radioactive colorimetric LDH assay and is based on the reduction of the tetrazolium salt MTT in a NADH-coupled enzymatic reaction to a reduced form of MTT which exhibits an absorption maximum at 565 nm. The intensity of the purple color formed is directly proportional to the enzyme activity.

### Sample Preparation:

Blood contacted fibers were washed thoroughly in phosphate buffered saline (PBS pH: 7.4) in order to remove loosely adhered platelets and thrombus. Fibers were homogenized in 1 mL buffer containing 100 mM potassium phosphate (pH 7.0) and 2 mM EDTA followed by centrifugation at 10,000 g for 15 min at 4° C. Supernatant contained eluted platelets from fibers were used for assay. Initially, known amounts of platelets from PRP as standards were mixed with the solution of 100 mM potassium phosphate (pH 7.0) and 2 mM EDTA in order to plot a standard linear curve of the assay. The working Reagent to indicate the LDH activity for this assay was prepared by mixing 14 μL MTT (tetrazolium dye) Solution, 8 μL NAD (nicotinamide adenine dinucleotide) Solution, 8 μL PMS (phenazine methosulfate) Solution and 170 μL Substrate Buffer.

100 μL samples of known amounts of platelets suspended in buffer solution were added to the 100 μL of working reagent into the sample wells and mixed thoroughly to notice a color change. Absorption was read OD565 nm (ODsO), and again after 25 min (ODs25) on a plate reader. Plotting absorption differences vs. platelets, results in a standard curve. This same procedure was repeated by replacing 100 mL of known platelets sample buffer with 100 mL of supernatant obtained after centrifugation of fiber samples within 1 ml of buffer. Comparing the absorption differences of samples with the absorption difference of standards through linear equation enables the quantification of adhered platelets.

### Scanning Electron Microscopy Imaging Characterization of HFMs Surfaces:

The PBS washed sample HFMs after the in-vitro experiments were fixed in general fixing solution containing glutaraldehyde and prepared for SEM imaging characterization. The samples were rinsed, dehydrated, critical point dried, mounted on metallic studs and sputter coated with Gold and Palladium. Samples were imaged with FEI Quanta 200 high performance thermal emission column scanning electron microscope. Distribution and morphological details of thrombus deposited on the surfaces of samples were characterized using the method of scanning electron microscopy (SEM) at an acceleration voltage of 4 kV and a working distance of 3 to 10 mm

### Results:

### Fibrinogen Adsorption to Test Surfaces of HFMs:

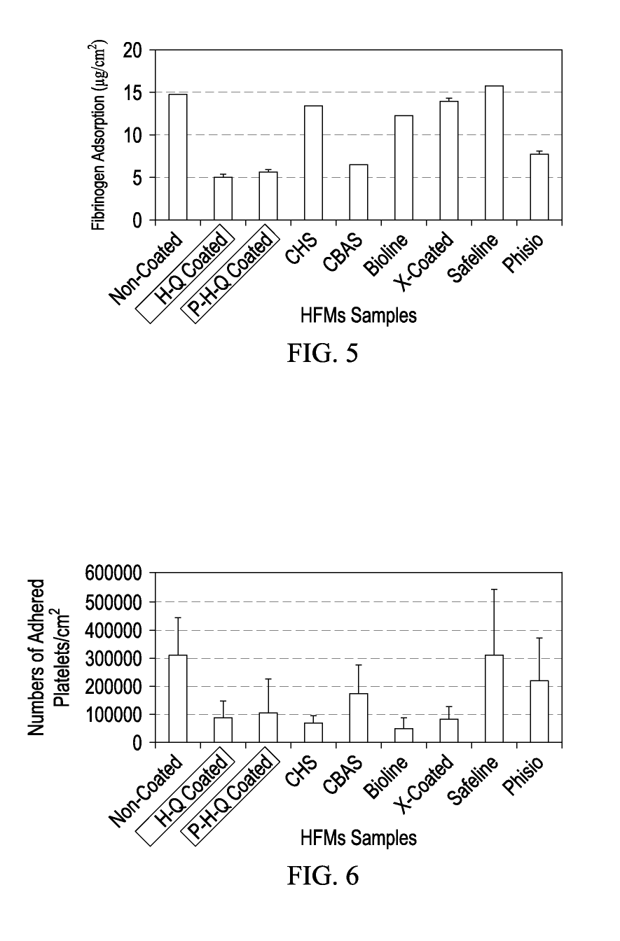

After the method of fibrinogen adsorption from a unit mg/ml in PBS solutions, the depleted contents of fibrinogen in PBS as an indication of absorption by HFMs surfaces are shown in FIG. 5. Both uncoated and SafeLine coated surfaces showed significant amounts of fibrinogen adsorption followed by X-coated, BioLine coated and CHS coated surfaces. New heparin coatings H-Q and P-H-Q of our laboratory showed improved resistance to fibrinogen by confining adsorption at one third of the protein approximately than that of the uncoated samples. CBAS and Phisio surfaces stood next to these new coatings.

### Platelet Adhesion to Test Surfaces:

Results of adhered platelets quantified using LDH activation studies are shown in FIG. 6. Platelets adhered to Non-coated samples are approximately of the order of 2.50×10⁵/cm² of the fiber surface. Significantly lower values of platelet adhesion are evident with the current QUAT and PVP coating samples and as well as commercial Bioline, CHS and X-Coating samples. Platelet adhesion to Safeline biopassive coated fibers is remarkable, where as those of SORIN and CBAS coated samples is appreciable and is in the range of 1.50×10⁵/cm² to 2.00×10⁵/cm². Both in-vivo and in-vitro studies of platelet adhesion from the literature (Anja K. Zimmermann et al., Effect of Biopassive and Bioactive Surface-Coatings on the Hemocompatibility of Membrane Oxygenators, J Biomed Mater Res Part B: Appl Biomater 80B, 2007, pp 433-439; Wendel HP, Ziemer G, Coating-techniques to improve the hemocompatibility of artificial surfaces used for extracorporeal circulation. Eur J Cardiothorac Surg 1999; 16: 342-350; Keuren J F, Wielders S J, Willems G M, Mona M, Lindhout T, Fibrinogen adsorption, platelet adhesion And thrombin gernation at heparinized surfaces exposed to flowing blood. Thromb Haemost 2002; 87: 742-747; Korn R L, Fisher C A, Livingston E R, Stenach N, Fishman S J, Jeevanadam V, Addonizio V P. The effects of Carmeda Bioactive Surface on human blood components during simulated extracorporeal circulation. J. Thorac Cardiovasc Surg 1996; 111: 1073-84) also indicate identical results for both bioactive and biopassive coatings.

### Scanning Electron Microscopy Imaging Characterization of HFMs Surfaces:

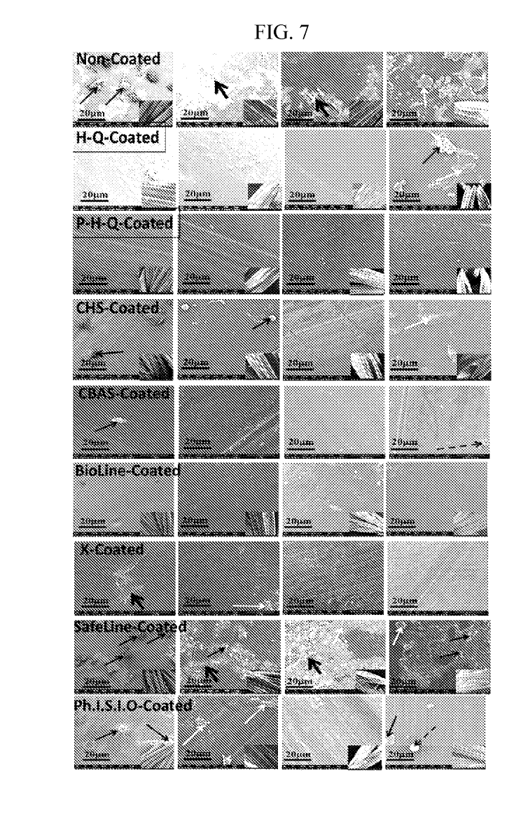

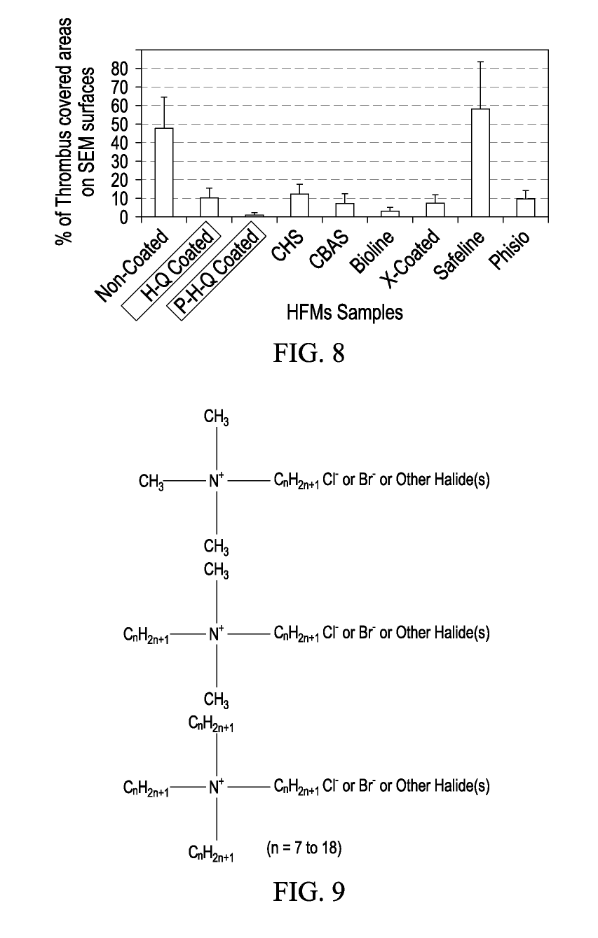

FIG. 7 shows the results of blood contacted fiber surfaces of the non-coated HFMs along with the current coatings and some commercial coatings from four independent in-vitro experiments. The results of current coatings are identified separately. In all four in-vitro experiments, non-coated fibers in contact with blood significantly encouraged protein adsorption and cell adhesion. Both QUAT-coated and PVP-QUAT coated fibers showed only nominal levels of thrombotic depositions on their surfaces. Activated platelets could be distinguished as separated, pseudopods formed and spherical morphology turned types during the first in-vitro experiment. FIG. 8 shows percentage of thrombotic depositions on the HFMs of all four in-vitro experiments that were estimated by quantifying and averaging SEM images of each surface (of 2000× magnification) as shown in FIG. 7. A transparent grid with 143 numbers of rectangles was applied to cover the entire surface of each SEM image. Total numbers of rectangles covering the thrombus were expressed as a percentage of whole units to describe the thrombus covered areas of each fiber surface. Mean values of thrombotic depositions along with respective standard deviations are reported for each type of uncoated or coated HFMs in this FIG. 8. Significant to insignificant levels of thrombus as in descending manner follows the trend as: ‘SafeLine>Uncoated>CHS Coated >H-Q Coated>Phisio Coated>X-Coated>CBAS Coated>BioLine>P-H-Q Coated’.

In the foregoing specification, the invention has been described with reference to specific embodiments thereof. It will, however, be evident that various modifications and changes may be made thereto without departing from the broader spirit and scope of the invention. Accordingly, it is intended that such changes and modifications fall within the scope of the present invention as defined by the claims appended hereto. The specification and drawings are, accordingly, to be regarded in an illustrative rather than restrictive sense.

We claim:

1. A biocompatible coating composition comprising
hemo-compatible, heparin-based bioactive coatings comprising quaternary ammonium salts and a heparin complex (QUAT); wherein said quaternary ammonium salts are selected from one or more long-chain linear quaternary aliphatic alkylammonium salts having at least one radical being a plain or substituted long-chain aliphatic group from C₇H₁₅ to C₁₈H₃₇.

2. The coating composition of claim 1, wherein said coating composition further comprises a hydrophilic priming layer.

3. The coating composition of claim 2, wherein said hydrophilic priming layer comprises polyvinylpyrrolidone (PVP).

4. A method of coating a Hollow Fiber Membrane (HFM) surface comprising
creating anionic functional groups on one or more surfaces of said HFM and thereafter coating the surface with a layer of said quaternary ammonium salts and heparin complex (QUAT), wherein said quaternary ammonium salts are selected from one or more long-chain linear quaternary aliphatic alkylammonium salts having at least one radical being a plain or substituted long-chain aliphatic group from C₇H₁₅ to C₁₈H₃₇.

5. The method of claim 4, wherein said anionic functional groups are created on the surfaces of said HFMs by modifying the surface of said HFM using ionic complexes dissolved in a solvent mixture; said solvent mixture comprising major quantity of alcohol along with a minor quantity of organic dissolving agents.

6. The method of claim 5, wherein said organic dissolving agent comprises one or more of Tetrahydrofuran (THF), Acetnone, Benzene, Toluene, Naphtha, Cyclohexane, n-heptane, n-hexane, Ether and Petroleum-ether.

7. The method of claim 6, wherein said solvent mixture comprising 70-100 vol. % Methanol and 30-0 vol. % of THF.

8. A method comprising
coating a surface with a priming solution comprising PVP and
thereafter coating said surface with a QUAT solution comprising quaternary ammonium salts and heparin; wherein said quaternary ammonium salts are selected from one or more long-chain linear quaternary aliphatic alkylammonium salts having at least one radical being a plain or substituted long-chain aliphatic group from C₇H₁₅ to C₁₈H₃₇.

9. The method of claim 8, wherein the priming solution comprises 0.5 to 0.9 wt. % of Poly-N-vinyl-2-pyrrolidone (PVP) dissolved in rest of wt. % of alcohol.

10. The method of claim 9, wherein said surface comprises one or more surfaces of Hollow Fiber Membrane (HFM).

11. A method for preparing non-thrombogenic bio-compatible coatings comprising:
mixing a solution of heparin dissolved in water with
a solution comprising hydrophobic Quaternary ammonium salts, or a mixture comprising one or more hydrophobic Quaternary ammonium salts with or without a mixture of one or more hydrophilic Quaternary ammonium salts dissolved in alcohol.

12. The method of claim 11, wherein said salts comprise one or more long-chain linear Quaternary aliphatic alkylammonium salts with at least one radical being a plain or substituted long-chain aliphatic group from C₇H₁₅ to Cₗ₈H₃₇.

13. The method of claim 11, wherein said mixing is done in a drop-wise fashion.

14. The method of claim 11, wherein said mixing produces a colloidal solution comprising a Quat (s)-Heparin complex and residual particles of heparin-rich Quat (s) and Quat (s)-rich heparin suspended within the solvent combination of water and alcohol; said method further comprising:
separating suspended precipitate and particles of solution in the form of a dense precipitate pellet using centrifugation and aliquot separation;
washing the dense precipitate with a fresh solvent mixture of deionized water and alcohol one or more times; and
discarding aliquot and expelling residual liquid from the complex.

15. The method of claim 14, wherein a complex obtained by the method is stored at below −20° C.

16. The method of claim 14, further comprising weighing the resulting complex and grinding the complex to fine particles and dissolving the complex in a solvent combination comprising 60 to 90 wt. % of alcohol with rest being at least one reagent.

17. The method of claim 16, wherein said reagent comprises at least one reagent selected from the group consisting of tetrahydrofuran (THF), Acetnone, Benzene, Toluene, Naphtha, Cyclohexane, n-heptane, n-hexane, Ether and Petroleum-ether.

18. A kit comprising
one or more hemo-compatible, anti- and/or non-thrombogenic, heparin-based bioactive coatings comprising a quaternary ammonium salt and heparin complex (QUAT) according to claim 1; and
at least one additional component selected from the group consisting of instructions for the use of such coatings; an oxygenator; and an artificial lung.

19. An oxygenator comprising one or more blood contacting surfaces coated with one or more coating according to claim 1.

20. A method comprising
filling a solution comprising 0.5 to 0.9 wt. % of Poly-N-vinyl-2-pyrrolidone (PVP) dissolved in 99.5-99.1 wt % of alcohol, through a blood inlet port of an oxygenator device;
rinsing with alcohol to remove PVP;
filling through the blood inlet port of the oxygenator device, a biocompatible coating composition comprising hemo-compatible, anti- and non-thrombogenic, heparin-based bioactive coatings comprising a quaternary ammonium salt and heparin complex (QUAT) dissolved in alcohol and THF solution, and contacting surfaces to be contacted with blood in the coating solution for 1 second to 10 minutes;
discarding and purging the coating composition out from the oxygenator device using inert gas; and
rinsing the oxygenator device with saline solution.
